(19) United States
(12) Patent Application Publication
(10) Pub. No.: US 2021/0030673 A1
(43) Pub. Date: Feb. 4, 2021

(54) Method and a Compound for Preventing Mammalian Cancer Cell Proliferation and for Treating Cancer

(71) Applicant: Oy Granula Ab Ltd., Kotka (FI)

(72) Inventors: Thomas AHLNÄS, Kotka (FI); Toveann AHLNÄS, Turku (FI)

(73) Assignee: Oy Granula Ab Ltd., Kotka (FI)

(21) Appl. No.: 16/945,991

(22) Filed: Aug. 3, 2020

Related U.S. Application Data
(60) Provisional application No. 62/981,608, filed on Feb. 26, 2020.
(60) Provisional application No. 62/881,975, filed on Aug. 2, 2019.

(51) Int. Cl. A61K 9/08; A61K 36/15; A61K 31/343; A61K 31/341; A61K 31/09; A61K 31/56

(52) U.S. Cl. CPC A61K 9/08; A61K 36/15; A61K 31/343; A61K 45/06; A61K 31/09; A61K 31/56; A61K 31/341

(57) ABSTRACT

A composition and a method for use in suppressing growth and/or proliferation of cancer cells in vivo and in vitro is provided. The composition includes an alcoholic lignan extract prepared from coniferous species optionally in combination with betulonic acid or betulinic acid. The composition inhibits growth and/or proliferation of at least prostate and melanoma cancer cells in vitro. Combination of the alcoholic lignan extract and betulonic or betulinic acid has a synergic effect specifically in inhibition of melanoma and prostate cancer cell proliferation and toxicity. Oral daily administration of the composition decreases or in some cases abolishes tumors of patients with breast cancer, brain cancer or colon cancer. The composition is suitable for use in cancer treatment in pet animals as well.

### PRIORITY

This application claims priority of provisional patent application No. 62/881,975 and 62/981,608 filed on Aug. 2, 2019 and Feb. 26, 2020, respectively. The contents of both of the applications are incorporated herein by reference.

### FIELD OF THE INVENTION

The invention generally relates to plant lignan extracts or mixtures of lignan extracts, and methods of using such extracts in treatments of cancer. More specifically the invention relates to lignan extracts from certain coniferous species for suppressing proliferation or growth of mammalian cancer cells. The invention further relates to use of a composition comprising a lignan extract or a mixture of lignan extracts from one or more of coniferous species in a method for suppressing cancer cell proliferation and/or growth in a mammal. The invention also relates to a method for suppressing proliferation of prostate and melanoma cancer cells in vitro, comprising a step of adding on the prostate or melanoma cell culture a composition comprising a lignan extract or a mixture of lignan extracts from one or more of coniferous species. Furthermore, the invention relates to synergistic effect of lignan extract and betulinic or betulonic acids in suppression of mammalian cancer cell proliferation.

### BACKGROUND OF THE INVENTION

Lignans are primarily structurally defined as polyphenols, which are complex molecules composed of a plurality of phenol molecules adhered and linked together and generally possess a 2,3-dibenzylbutane structure. They include, among other compounds the following but not limited to matairesinol, conidendrin, secoisolariciresinol, lariciresinol, isolariciresinol, nordihydroguaiaretic acid, pinoresinol, liovil and olivil, pinoresinol, nortrachelogenin, matairesinol and juvabiones and other compounds, which may be precursors of enterolactone and enterodiol and modifications thereof, including diglucosides.

Plant lignans are the primary source of dietary phytoestrogens in modern diet. Phytoestrogens have multitude beneficial health-effects; high levels of lignans can support for example healthy glucose metabolism and can further reduce insulin sensitivity. Plant lignans have been suggested to be used for treating general infections and various inflammatory conditions; for example, U.S. Pat. No. 5,762,935 discloses anti-inflammatory effects of lignans from sesamin family.

However, edible plants (fruits, vegetables and seeds) contain considerably low levels of lignans, such that their health effects in normal diet is almost insignificant and hardly can be taken into account. Wood lignans, on the other hand, are abundantly found in wood material and in bark, and as such, one might consider using these lignans in the diet in the same way as plant lignans from fruits and seeds. Thus, it has been suggested to use wood lignans in food products. U.S. Pat. No. 10,172,896 discloses a method to lower cholesterol level in blood serum of a mammal by orally administering a composition of lignan extract of coniferous species.

Epidemiological studies relating diet to disease suggest that dietary components may predispose populations to reduced risk of certain diseases. Far eastern populations consuming soy, for example, exhibit reduced rates of breast, prostate and colon cancers and coronary heart disease, while populations, for example, in Finland have increased rates of prostate cancer. Researchers are now just studying, and consequently, realizing the specific compounds in the diet, in order to put the puzzle together, to better understand the basis for the epidemiological observations.

Superior results may be obtainable when a plurality of phytochemicals is consumed in combinations. Therefore, a proper diet containing a combination of the desired phytochemicals, may be a desirous way to prevent various cancer forms. A problem in today's world is that a large part of population either does not have access to proper kind of diet, or simply chooses not to follow such diet. Accordingly, there is an unmet need for dietary supplements for improving life quality of general public.

Currently, there is almost an epidemic of cancer world-wide. Traditional medical treatments include surgeries, chemotherapy, radiation, and heavy medications. On the other hand, there is plenty of showing that many cancer forms could be prevented by lifestyle choices, including dietary choices. Accordingly, the unmet need for dietary supplements for improvement of life quality is applicable also in the area of prevention and treatment of cancers of various kinds. In the modern world cancer has become common not only in human beings but also in pet animals. Accordingly, there is a further need for a dietary supplement for pet animals as well.

### SUMMARY OF THE INVENTION

The invention according to this disclosure provides solutions to the above-mentioned shortcomings or at least alleviate above mentioned problems.

Therefore, it is an object of this invention to provide a composition comprising a lignan extract or a mixture of lignan extracts from one or more of coniferous species selected from genus Picea, Abies, and Pinus, and comprising oligolignans, and at least the following lignans: 7-hydroxymatairesinol, conidendrin, lariciresinol, liovil and secoisolariciresinol or their geometric isomers or stereoisomers for use in a method for suppressing mammalian cancer cell proliferation or growth in vitro or in vivo. It is an object of this invention to provide such composition for suppressing proliferation of cancer cells in vitro and in vivo.

It is another object of this invention to provide a composition comprising mixture of lignan extracts from one or more of coniferous species selected from genus Picea, Abies, and Pinus, together with a low concentration of betulinic or betulonic acid for use in a method for improved suppression mammalian cancer cell proliferation in vitro or in vivo. It is an object of this invention to provide such composition for suppressing proliferation of melanoma cells in vitro and in vivo.

It is further an object of this invention to provide a composition for use in a method for suppressing mammalian cancer cell proliferation or growth in vitro, wherein the composition comprises 5-50 wt % of the lignan extract and proportion of said lignans in the lignan extract is: 7-hydroxymatairesinol 70-80 wt %; conidendrin 3-8 wt %; lariciresinol 1-4 wt %; liovil 2-5 wt %; secoisolariciresinol 3-7 wt %; and other lignans 0-3 wt %, wherein the fraction of other lignans includes various oligolignans, and optionally betulinic acid or betulonic acid.

It is an object of this invention to provide a composition comprising a lignan extract and optionally betulonic acid or betulinic acid for use in a method to suppress cancer cell proliferation or growth in a mammal having been diagnosed with melanoma, a brain cancer tumor, a colon cancer tumor, a breast cancer tumor, or a prostate cancer tumor.

Another object of the invention is to provide a use of a composition comprising a lignan extract or a mixture of lignan extracts from one or more of coniferous species selected from genus Picea, Abies, and Pinus and optionally betulonic acid or betulinic acid for suppressing cancer cell proliferation and/or growth in a mammal, wherein said extract or mixture of extracts comprises at least the following lignans: 7-hydroxymatairesinol, conidendrin, lariciresinol, liovil and secoisolariciresinol or their geometric isomers or stereoisomers, as well as a fraction containing various oligolignans.

It is yet another object of the invention to provide a method of suppressing cancer cell proliferation or growth in a mammal, said method comprising oral administration of a composition comprising a lignan extract or a mixture of lignan extracts from one or more of coniferous species selected from genus Picea, Abies, and Pinus and optionally betulonic acid or betulinic acid, wherein said extract or mixture of extracts comprises at least the following lignans: 7-hydroxymatairesinol, conidendrin, lariciresinol, liovil and secoisolariciresinol or their geometric isomers or stereoisomers, as well as a fraction containing various oligolignans.

It is yet another object of the invention to provide a composition for use in preparing a medicament for suppressing mammalian cancer cell proliferation or growth, wherein said composition comprises a lignan extract or a mixture of lignan extracts from one or more of coniferous species selected from genus Picea, Abies, and Pinus and optionally betulonic acid or betulinic acid, wherein said extract or mixture of extracts comprises at least the following lignans: 7-hydroxymatairesinol, conidendrin, lariciresinol, liovil and secoisolariciresinol or their geometric isomers or stereoisomers, as well as a fraction containing various oligolignans.

It is an object of this invention to provide a method to suppress proliferation or growth of melanoma cells, brain cancer cells, colon cancer cells, breast cancer cells, and prostate cancer cells by orally administering a lignan extract to a mammal.

It is a further object of this invention to provide a method for suppressing proliferation or growth of prostate and melanoma cancer cells in vitro, comprising a step of treating said prostate and melanoma cells with a composition, comprising a lignan extract or a mixture of lignan extracts from one or more of coniferous species selected from genus Picea, Abies, and Pinus and optionally betulonic acid or betulinic acid and comprising at least the following lignans: 7-hydroxymatairesinol, conidendrin, lariciresinol, liovil and secoisolariciresinol or their geometric isomers or stereoisomers, as well as a fraction containing various oligolignans.

Another object of the invention is to provide a method for preparing a composition for treating melanoma or prostate cancer cells, said method comprising following steps: extracting alcoholic solution of 5-50 wt % of the lignan extract or the mixture of extracts with a lower alcohol from one or more of coniferous species selected from genus Picea, Abies, and Pinus; and admixing betulonic acid or betulinic acid preferably obtained from bark of Betula pendula into the alcoholic solution of lignan extract.

Yet another object of the invention is to provide a composition for topical use in treating or preventing burning injuries and skin injuries after radiation treatments.

### BRIEF DESCRIPTION OF THE DRAWINGS

The following Figures form part of the present specification and are included to further demonstrate certain aspects of the present invention. The invention may be better understood by reference to one or more of these Figures in combination with the detailed description of the specification embodiments presented herein.

FIG. 1A shows PC-3 prostate cancer cells 48 hours after treatment with a corresponding 24 μM 1,3-propanediol control.

FIG. 1B shows PC-3 prostate cancer cells treated with 24 μM lignan extract.

FIG. 1C shows untreated control of PC-3 cells containing only growth medium. All images were taken with a 5× magnification.

FIG. 2A shows PC-3 prostate cancer cells treated with corresponding 24 μM 1,3-propanediol control.

FIG. 2B shows untreated control of PC-3 cells containing only growth medium.

FIG. 2C shows PC-3 prostate cancer cells 48 hours after treatment with 24 μM lignan extract of this disclosure. All images were taken with a 20× magnification.

FIG. 3A shows live-cell imaging of PC-3 prostate cancer cell proliferation during a 48-hour treatment with different concentrations of a lignan extract of this disclosure or control exposure. Y-axis represents the confluency of cells per well (20000 cells at seeding), meaning the percent of well surface covered in adherent cells. Each point on the graph is the mean confluency of triplicate samples on a 24-well plate. PC-3 cells were either treated with 24 μM, 36 μM, or 60 μM 1,3-propanediol as solvent control, or with 24 μM, 36 μM or 60 μM lignan extract, or with an untreated control comprising only the growth medium.

FIG. 3B shows selectively the same data as FIG. 3A. Now only treatment with the lignan extracts (24 μM, 36 μM, and 60 μM) and the untreated controls are shown. Statistical analysis using one-way ANOVA and Dunette's multiple comparisons test done with GraphPad Prism 6 software of the data at the exposure time of 48 hours indicated significant differences between untreated and 60 μM extract treated PC-3 cells (P<0.05).

FIG. 3C shows cell viability of serum-starved PC-3 cells measured after 24-hour treatment with different concentrations of lignan extract from Picea abies. IC₅₀-value in these treatment conditions was measured to equal 20.02 μM using GraphPad Prism 6 software.

FIG. 4A shows SK-Mel-5 melanoma cancer cells 48 hours after treatment with 24 μM corresponding 1,3-propanediol control.

FIG. 4B shows SK-Mel-5 melanoma cancer cells treated with 24 μM lignan extract.

FIG. 4C shows a control of SK-Mel-5 cells in growth medium. SK-Mel-5 melanoma cancer cells proliferate at a high rate both in the lignan extract-treated sample and in controls. No significant effect was observed with the lignan exposure. All images were taken with a 5× magnification.

FIG. 5A shows SK-Mel-5 melanoma cancer cells 48 hours after treatment with 24 μM corresponding 1,3-propanediol.

FIG. 5B shows SK-Mel-5 melanoma cancer cells treated with 60 μM lignan extract.

FIG. 5C shows untreated SK-Mel-5 cells in growth medium. SK-Mel-5 melanoma cancer cells proliferate at a high rate both in the treated sample and when treated with the controls. No significant effect was observed with the lignan exposure. All images were taken with a 20× magnification.

FIG. 6A shows live-cell imaging of WM-266-4 melanoma cancer cell proliferation during a 48-hour treatment with different concentrations of the lignan extract or control exposure. Y-axis represents the confluency of cells per well (30000 cells at seeding), meaning the percent of well surface covered adherent cells. Each point on the graph is the mean of triplicate samples on a 24-well plate. WM-266-4 melanoma cancer cells were either treated with 24 μM, 36 μM, or 60 μM propanediol control, or 24 μM, 36 μM or 60 μM lignan extract, or with an untreated control comprising only the growth medium.

FIG. 6B shows selectively the same data as FIG. 6A. Now only treatment with the lignan extracts (24 μM, 36 μM, and 60 μM) and the untreated controls are shown. Statistical analysis using one-way ANOVA and Dunette's multiple comparisons test done with GraphPad Prism 6 software of the data at the exposure time of 48 hours indicated significant differences between untreated and 60 μM extract treated WM-266-4 cells (P<0.05).

FIGS. 7A-7D illustrate percentual proportion of PC-3 cells in various stages of the cell cycle after a 24-hour treatment with lignan extracts or controls (±SEM). Statistical analysis was carried out by one-way ANOVA followed by Tukey's multiple comparison test using GraphPad Prism 6 software. *** and **** represent a significance value where P<0.001 and P<0.0001, respectively. FIG. 7A shows the percentual proportion of PC-3 cells in G0/G1-phase after a 24-hour treatment with growth medium only (control), with 24 μM lignan extract, and with 60 μM lignan extract. FIG. 7B shows the percentual proportion of PC-3 cells in S-phase after a 24-hour treatment with growth medium only (control), with 24 μM lignan extract, and with 60 μM lignan extract. FIG. 7C percentual proportion of PC-3 cells in G2/M-phase after a 24-hour treatment with growth medium only (control), with 24 μM lignan extract, and with 60 μM lignan extract. FIG. 7D represents flow cytometry results from one individual example experiment with propidium iodide stained PC-3 cells treated for 24 hours with growth medium only (control; the lowermost curve); cells treated with 24 μM extract for 24 hours (middle curve) and with 60 μM extract for 24 hours (uppermost curve). Significant differences in the proportion of cells in G0/G1-phase and G2/M-phase can be observed when comparing the curves, indicating that the treated cells are accumulating in G0/G1-phase of the cell cycle.

FIG. 8A shows live-cell imaging of normal human dermal fibroblast (HDF) cell proliferation during 48 h treatment with betulinic acid (BA). Results are mean values from three independent experiments (±SEM).

FIG. 8B shows live-cell imaging of normal human dermal fibroblast (HDF) cell proliferation during 48 h treatment with betulonic acid (BOA). Results are mean values from three independent experiments (±SEM).

FIG. 9A shows live-cell imaging of metastatic prostate cancer (PC-3) cell proliferation during 48 h treatment with betulinic acid (BA) (±SE).

FIG. 9B shows live-cell imaging of metastatic melanoma (WM-266-4) cell proliferation during 48 h treatment with betulonic acid (BOA) (±SE).

FIG. 9C shows live-cell imaging of metastatic prostate cancer (PC-3) cell proliferation during 48 h treatment with betulonic acid (±SE).

FIG. 10A shows cytotoxicity assay of betulinic acid (BA) on metastatic melanoma (WM-266-4) cells after 48 h treatment (±SEM). Results are mean values from three independent experiments.

FIG. 10B shows cytotoxicity assay of betulinic acid (BA) on metastatic prostate cancer (PC-3) cells after 48 h treatment (±SEM). Results are mean values from three independent experiments.

FIG. 11A shows cytotoxicity assay of betulonic acid (BOA) on metastatic prostate cancer (PC-3) cells after 48 h treatment (±SEM). Results are mean values from three independent experiments.

FIG. 11B shows cytotoxicity assay of betulonic acid (BOA) on metastatic melanoma (WM-266-4) cells after 48 h treatment (±SEM). Results are mean values from three independent experiments.

FIG. 12A shows live-cell imaging of cell proliferation of PC-3 cells treated with P. abies knotwood extract with or without betulonic acid (BOA) for 48 hours. Values represent two independent experiments (±SEM).

FIG. 12B shows live-cell imaging of cell proliferation of PC-3 cells treated with P. abies knotwood extract with or without betulinic acid (BA) for 48 hours. Values represent two independent experiments (±SEM).

FIG. 12C shows live-cell imaging of cell proliferation of WM-266-4 cells treated with P. abies knotwood extract with or without betulonic acid (BOA) for 48 hours.

FIG. 12D shows live-cell imaging of cell proliferation of WM-266-4 cells treated with P. abies knotwood extract with or without betulinic acid (BA) for 48 hours.

### DETAILED DESCRIPTION OF THE INVENTION

The present invention is based on the surprising observation that a certain mixture of lignans in an extract, including at least: 7-hydroxymatairesinol, conidendrin, lariciresinol, liovil and secoisolariciresinol or their geometric isomers or stereoisomers thereof, as well as a fraction containing various oligolignans, has a suppressing effect on the proliferation or growth of human prostate and melanoma cancer cells, in vitro.

Further studies showed that the suppression specifically took place during the G0/G1-phase of the cell cycle.

This observation was then applied to preparation of a supplement for oral use of human beings as well as pet animals. The results showed that daily intake of the composition indeed reduced existing tumors and in some cases completely abolished the tumors resulting the patients to get ‘clean papers’ from their doctors.

A further surprising result of experiments was that betulonic or betulinic acid in low concentrations had a synergistic effect with the lignan extract in suppressing the proliferation of growth of human prostate and melanoma cancer cells in vitro. Adding betulinic or betulonic acid into the preparation of the lignan mixture supplement is expected to have at least as good and likely better effect on reducing existing tumors as the lignan mixture supplement alone.

The various particular embodiments of the present invention are surprising, because the combination of a mixture of wood lignans and oligolignans, has not been known to have an effect on the proliferation and growth of cancer cells in vitro, nor has it been suggested that use of a mixture of wood lignans and oligolignans could be used in methods to abolish tumor growth in mammals. Even if there are some studies showing betulonic and betulinic acid derivatives having effects on cancer cells, the strong synergistic effect of the lignan mixture and betulonic or betulinic acid reducing growth rate of cancer cells has never been reported.

Preparation of the Composition

The composition of the present invention is based on the fact that the phenolic compounds, such as lignans, tannins and flavonoids contained in the wood material, are extracted from the wood with an extraction medium, which as such, is suitable for various food products. Extraction medium may be an extraction solution or subcritical or supercritical extraction fluid, such as CO₂. In the previous practice of the art, lignans, tannins and flavonoids have been purified before using them in food industry.

Different from the previous practice exploited, the particular embodiments of the present invention, is based on the fact, that the raw extraction solution or extraction fluid is not purified before use. The raw extraction solution, i.e. wood extract or raw extraction fluid-wood extract is recovered from the wood material, such as wood chips or chipped wood pulp of the chemical pulp industry, which contain(s) lignans and/or flavonoids and/or tannins as active ingredients and are/is then subsequently used for as a dietary supplement, as a composition for further preparing medicaments or natural treatments, or alternatively as an additive in various food products, or semi-finished food products.

In a case, this composition according to the particular embodiments of the present invention being a solution composed of liquid-wood extract, the relative amounts of the active chemical compositions in the lignan mixture may be modified for better suiting in a designed use. This can be obtained, for example by a fractionating extracting procedure. The solvent to be used as an extraction solution may be simply selected on the basis of its suitability to further uses. This provides the advantage that the isolation of the raw extraction solution—wood extract or raw extraction fluid-wood extract from the wood material is simple and requires a considerably smaller number of process stages than before.

Preferably the lignan mixture used in the products and compositions according to the particular embodiments of the present invention is obtained by extracting or grinding wood bark and/or knot wood from coniferous species from the genus Picea and/or Abies. According to certain embodiments also species from the genus Pinus may be used. According to certain embodiments also species from the genus Betula may be used.

An extract from Picea abies (Norwegian spruce tree) is one preferable embodiment and the lignan extract obtained from knot wood of Norwegian spruce tree has the following proportions of lignans: 7-hydroxymatairesinol 70-80 wt %; conidendrin 3-8 wt %; lariciresinol 1-4 wt %; liovil 2-5 wt %; secoisolariciresinol 3-7 wt %, other lignans 0-3 wt %.

Usually, this lignan mixture may also contain oligolignans and the fraction of ‘other lignans’ contains various oligolignans. Into this lignan mixture betulonic or betulinic acid, or both of them may also be added. Betulonic or betulinic acid may originate from lignan extract from Betula species.

Depending on the source of wood material, the extract may also contain other wood Zs lignans, such as but not limited to, pinoresinol, nortrachelogenin, matairesinol and juvabiones.

In some particular embodiments, the raw extraction solution-wood extract may preferably be from a single tree species, especially from spruce tree (Picea sp). In some particular embodiments, the tree may be the Norwegian spruce tree (Picea abies L). In other particular embodiments, a mixture that originates from two or more tree species may equally, and advantageously be applied, exemplary beneficial mixture originating from a spruce (Picea sp.) and/or fir (Abies sp.) and a pine (Pinus sp.) or from a conifer and a birch (Betula sp.). According to certain embodiments a mixture is made of multiple wood extracts. According to certain embodiments one extract is received from a mixture of wood chips of more than one species. According to a preferred embodiment the extract is received from a mixture of wood chips of two species; according to a preferred embodiment the species are Picea abies and Pinus sp. (e.g. Pinus sylvestris L.). According to one preferred embodiment the species are Picea abies and Betula sp. According to one preferred embodiment the species are Pinus sp. and Betula sp. According to certain embodiments the mixture is obtained from more than two species; according to certain embodiments the mixture is obtained from Picea abies, Pinus sp. and Betula sp. In certain preferred embodiments, the wood may originate from trees belonging to the genus Picea or the genus Abies. According to certain embodiments the mixture is prepared of lignan extracts from Picea species and from Abies species. According to certain embodiments the mixture is prepared from lignan extracts from Picea sp. and/or Abies sp. and of Pinus sp. and/or Betula sp. In certain other particular embodiments, the wood extract may originate specifically from Picea sp., Tsuga sp. (hemlock) or Acacia sp. In still other particular embodiments, the wood extract may additionally originate from bark of Betula sp., preferably Betula pendula (European white birch).

The types and amounts of mixture of lignan extracts obtained from different wood species vary considerably. By adding, and thereby combining, lignan extracts from various wood species, or plants, one might modify the altogether effect synergistically of the lignan mixture (i.e. active ingredient). As is shown here, adding betulinic or betulonic acid to lignan extract from P. abies has a clearly synergistic effect in vitro on prevention of cell proliferation of metastatic prostate cancer or melanoma cells.

For extraction of knot wood and/or bark with an extraction solution, for obtaining an alcohol-wood extract or an alcohol-water-wood extract, various alcohol-based solvents, such as but not limited to, butylene glycol, butylene glycol+monoalcohol, glycerol or glycerol+alcohol may be used. These alcohol-based solvents are either physiologically acceptable or/and edible.

The extraction solution may preferably contain a lower monovalent or bivalent alkyl alcohol or a mixture of a lower monovalent and a bivalent alkyl alcohol and/or glycerol, or a mixture of a lower (monovalent) alkyl alcohol and a bivalent or a trivalent lower alkyl alcohol. The monovalent alcohol may advantageously be ethanol, propanol, butanol, heptanol, octanol or decanol. The monoalcohol may especially and preferably be ethanol. The bivalent alkyl alcohol may preferably be a lower alkylene glycol, which may preferably be selected from the group consisting of ethylene glycol, propylene glycol, butylene glycol, pentylene glycol, and dipropylene glycol. Butylene glycol is an especially preferable solvent, as the lignan mixture according to particular embodiments of the present the invention dissolves therein in amounts of over 10% and it is physiologically tolerable.

Solutions of ethanol and bivalent alcohols are preferable for dissolving and also for extracting the lignan mixtures according to the particular embodiments of the invention. Bivalent alcohol may especially and preferably be selected from the group composing of ethylene glycol, propylene glycol, butylene glycol, pentylene glycol, and dipropylene glycol.

Suitable trivalent alcohol solvents may, for example, be glycerols.

The mixture of the lower alkyl alcohol and glycerol or alkyl glycol is a preferable solvent medium, as the need to further process the raw extracts (=solvent+active ingredient mixture) or raw extract concentrates based on extraction solutions containing such alcohols is as small as possible, when preparing different food additives or food grade compositions.

The alcohol or water-alcohol based raw alcohol-wood extracts or alcohol-wood extracts are well-suited to the manufacture of food-grade components or to be used as such. These food grade components include, without limiting to them, emulsions, dispersions, oils, sweeteners.

The present lignan mixture of the particular embodiments of the present invention may also be mixed with carrier or extraction solvents, which are appropriate food additives. The lignan mixture can be either powder or alcohol-water-wood extract or simply alcohol-wood extract. These carrier or extraction solvents may include the following, but not limited to: acetone, benzyl alcohol, 1,3-butylene glycol, carbon dioxide, castor oil, citric acid esters of mono- and diglycerides, ethyl acetate, ethanol, glycerol (glycerin), glyceryl diacetate, glyceryl triacetate, glyceryl tributyrate, hexane, isopropanol alcohol, methyl alcohol, methyl ethyl ketone(2-butanone), methylene chloride, mono- and diglycerides, monoglyceride citrate, 2-nitropropane, 1,2-propylene glycol (1,2-propanediol), propylene glycol monoesters and diesters of fat-forming fatty acids, triethyl citrate.

Certain particular embodiments of the present invention are related to compositions, and use of such compositions in methods for suppressing proliferation or growth of cancer cells in vitro or in vivo, of at least of prostate and melanoma cancer cells, but also brain cancer cells, colon cancer cells, and breast cancer cells, where the compositions are lignan extracts comprising at least 7-hydroxymatairesinol, conidendrin; lariciresinol; liovil; and secoisolariciresinol. Certain particular embodiments of the present invention also comprise a fraction containing various oligolignans. Certain particular embodiments comprise adding betulonic or betulinic acid in low concentrations to the compositions of lignan extracts comprising at least 7-hydroxymatairesinol, conidendrin, lariciresinol, liovil, secoisolariciresinol and optionally also various oligolignans.

Certain particular embodiment of the present invention are related to compositions, and use of such compositions in methods for suppressing proliferation or growth of cancer cells in vitro or in vivo, of at least of prostate and melanoma cancer cells but also brain cancer cells, colon cancer, and breast cancer cells, where the compositions are lignan extracts comprising 7-hydroxymatairesinol 70-80 wt %; conidendrin 3-8 wt %; lariciresinol 1-4 wt %; liovil 2-5 wt %; secoisolariciresinol 3-7 wt %, other lignans 0-3 wt %, and optionally betulonic or betulinic acid.

Certain particular embodiments of the present invention are related to compositions, and use of such compositions in methods for suppressing proliferation or growth of cancer cells in vitro or in vivo, of at least of prostate and melanoma cancer cells but also brain cancer cells, colon cancer, and breast cancer cells, where the compositions are lignan extracts including additionally one or more lignan(s) at a range of 0-10 wt % selected from the group consisting of: pinoresinol, nortrachelogenin, matairesinol, juvabiones, or their geometric isomers or stereoisomers thereof and oligolignans. Into the compositions may further be added betulonic and/or betulinic acid to achieve synergistic effects in cell proliferation.

Certain particular embodiments of the present invention are related to compositions, and use of such composition in methods for suppressing proliferation or growth of cancer cells in vitro or in vivo, of at least of prostate and melanoma cancer cells but also brain cancer cells, colon cancer, and breast cancer cells, where the compositions are lignan extracts and the composition further include stilbenes or triterpens selected from the group consisting of betulin, betulonic acid, betulinic acid, betuloinic acid, resveratrol, their geometric isomers and stereoisomers thereof and oligolignans. Into the compositions may further be added betulonic and/or betulinic acid to achieve synergistic effects in cell proliferation.

Certain particular embodiments of the present invention are related to compositions and use of such compositions in methods for suppressing proliferation or growth of cancer cells in vitro or in vivo, of at least of prostate and melanoma cancer cells but also brain cancer cells, colon cancer, and breast cancer cells, where the compositions are lignan extracts from coniferous species such as Picea sp. or Pinus sp., with optionally added betulonic or betulinic acids preferably extracted from Betula sp. and the composition further includes comprises vitamins or micronutrients.

Certain particular embodiments of the present invention are related to a method for preparing a composition and use of a lignan extract or a mixture of lignan extracts for suppressing cancer cell growth and/or proliferation, in vitro or in vivo, or for preparation of treatments to suppress cancer cell growth and/or proliferation, where the composition includes at a range of 1-50 wt %, preferably 5-50 wt % of a lignan mixture including at least the following lignans: 7-hydroxymatairesinol, conidendrin, lariciresinol, liovil, secoisolariciresinol or their geometric isomers or stereoisomers thereof as well as a fraction containing various oligolignans and optionally added betulonic or betulinic acids, which method, includes at least the following steps:

a) extracting the mixture of lignans from a wood bark and/or knot wood from one or more coniferous species selected from genus Picea, Abies, and Pinus, with an extraction solution selected from the group consisting of an alcohol and an alcohol-water mixture;

b) obtaining an alcohol-wood lignan mixture or an alcohol-water-wood lignan mixture;

c) optionally adding betulonic or betulinic acid preferably extracted from Betula sp. and added as an alcohol mixture, and

d) adding a physiologically and pharmaceutically acceptable carrier agent to the mixture.

According to certain embodiment the composition for use in prevention of cancer cell growth and/or proliferation comprises 1-50 wt % of a lignan mixture of the total composition. More preferably, the amount of the lignan extract is about 5-50 wt % of the total composition.

According to certain embodiment the composition has 10-100 μM betulinic acid or betulonic acid. More preferably the composition has 10-60 μM betulinic acid or betulonic acid. Most preferably the composition has 20-40 μM betulinic acid or betulonic acid. A preferred concentration is approximately 30 μM betulonic or betulinic acid in the extract. According to certain embodiment the composition comprising 1-50 wt % of a lignan extract with optional addition of betulonic or betulinic acid can be used for preventing cancer cell growth and/or proliferation in relatively small quantities. According to certain embodiments a daily dosage of the extract amounts to 20 to 250 mg, more preferably to 50 to 200 mg and most preferably to 100-200 mg of 7-hydroxymatairesinol along with other lignans selected from the group consisting of conidendrin, lariciresinol, liovil and secoisolariciresinol and oligolignans and optionally betulonic or betulinic acid in a concentration of about 30 μM.

According to certain embodiment the daily dosage of the extract is such that it contains 100-200 mg of 7-hydroxymatairesinol, 4-8 mg each of the following lignans scoisolariciresinol, condendrin, liovil, lariciresinol, and 4-20 mg other lignans including oligolignans.

The embodiments of the present invention relate to the use of a composition including at a range of 1-50 wt %, preferably at a range of 5-50 wt % of a lignan mixture including at least following lignans: 7-hydroxymatairesinol, conidendrin, lariciresinol, liovil and secoisolariciresinol or their geometric isomers or stereoisomers thereof along with oligolingnans and optionally betulonic or betulinic acid for lowering, suppressing, and thereby inhibiting, in vitro, the proliferation and growth of mammalian cancer cells. As used herein this disclosure, the percent ranges given for the lignan mixture specifically refers to percent by weight of the total composition.

To be more exact, the particular embodiments of the present invention relate to i) a composition, ii) methods of preparing the composition, and iii) methods of using the composition for suppressing, and thereby, inhibiting the growth and/or proliferation of at least prostate and melanoma cancer cells, in vitro. This includes a lignan mixture including at least the following lignans: 7-hydroxymatairesinol, conidendrin, lariciresinol, liovil and secoisolariciresinol or their geometric isomers or stereoisomers thereof and a fraction including oligolignans and optionally betulonic or betulinic acid in a combination with an extraction solution, selected from the group consisting of alcohol and water-alcohol mixture, the balance being a physiologically acceptable carrier agent.

According to certain embodiments, the in vitro activity of lignan extracts may be used in selecting proper lignan extract with specific lignan concentration for preparation a treatment for use in suppressing tumor growth in a patient diagnosed with tumor.

One essential aspect of the particular embodiments of the present invention is also to make wood lignans available, in such a level, in an edible food product or as a supplement that their concentration is high enough so as to ensure, that their beneficial cancer abolishing lowering effect will appear. Normal wood material contains only small amounts of wood lignans and making food supplements from this kind of wood material would be cumbersome and ineffective. However, wood bark and/or knot wood have been found to include much higher amounts of these lignans in order to make the aforementioned lignan mixture possible.

Therefore, another essential aspect of the present invention relates to a method for preparing a composition for use in suppressing, and thereby eradicating cancer cells, including prostate and melanoma cancer cells by using wood bark and/or knot wood, as a source of obtaining the lignans. The method for making the compositions includes a) extracting the lignan mixture from a wood bark and/or knot wood from one or more coniferous species selected from at least the genus Picea and Abies, with an extraction solution selected from the group consisting of an alcohol and an alcohol-water mixture; b) obtaining a mixture of alcohol-wood lignan mixture or an alcohol-water-wood lignan; c) optionally adding betulinic or betulonic acid, and d) adding a physiologically and pharmaceutically acceptable carrier agent to said mixture.

The present invention also relates to a food additive, nutraceutical or dietary supplement for use in methods for suppressing the growth of tumors, including colon cancer tumors, brain cancer tumors, prostate cancer tumors, and breast cancer tumors of mammals.

Particular embodiments of the present invention relate to a use of a composition as a dietary supplement or an additive for a food product capable of suppressing the growth and/or proliferation of cancer cells and tumor growth in vivo, the composition comprising at a range of 1-50 wt %, preferably 5-50 wt % a lignan mixture including at least following lignans: 7-hydroxymatairesinol, conidendrin, lariciresinol, liovil and secoisolariciresinol or their geometric isomers or stereoisomers thereof as well as a fraction of oligolignans and optionally betulonic and/or betulinic acid in combination with an edible solid or liquid carrier material.

Preferably, the composition may include at a range of 1-90 wt %, preferably at a range of 5-90 wt %, the lignan mixture in a combination with an extraction solution selected from the group consisting of alcohol and water-alcohol mixture, the balance being an edible solid or a liquid carrier material.

The spectrum of effect of lignan mixture can be widened by adding other wood lignans or triterpenes having for example different antimicrobial potency than the mentioned lignan mixture. As is shown here, adding low concentration of betulonic or betulinic acid provides an improved reduction of cancer cell proliferation.

The composition may preferably be formulated, such that, the lignan mixture and a carrier material form together an encapsulated liquid, powder or pill, or a solution or a suspension.

According to certain embodiments the compositions of this disclosure may be used in combination with various medical cancer treatments, such as radiation- or chemotherapy. In certain embodiments, the compositions are used during the radiation- or chemotherapy treatment; in certain other embodiments the composition are used after cessation of radiation- or chemotherapy.

Certain further embodiments are related to a method for decreasing the risk of cancers in a mammal in general, the method including administering orally a dietary supplement of any one of the previously mentioned embodiments to a mammal on daily basis.

### Definitions

Unless defined otherwise, all technical and scientific terms used herein have the same meaning as commonly understood by one of ordinary skill in the art to which this invention belongs. All technical and scientific terms used herein have the same meaning. Although any materials similar or equivalent to those described herein can also be used in the practice of the present invention, exemplary materials are described for illustrative purposes.

As used herein and in the appended claims, the singular form “a”, “and”, “the” include plural referents unless the context clearly dictates otherwise. All technical and scientific terms used herein have the same meaning.

As used herein this disclosure, the terms “weight-percent” and “wt %” specifically refer to the individual weight of a component or a constituent, taken in relation to the entire total weight of a formulation, unless stated and specified otherwise, in which case, it would be intended to mean the individual weight of the component or the constituent relative to the total volume of the formulation.

As used herein this disclosure, the terms “composition”, “dietary composition”, or “pharmaceutical composition”, are used interchangeably, and hence, they all refer to a composition of matter essentially having a mixture of lignans.

As used herein, the term ‘synergistic’ or ‘synergistically’ refer to effect of two or more substances to produce a combined effect greater than the sum of their separate effects.

As used herein, the terms “about” and “approximately” are used interchangeably, and are meant to designate any value which lies preferably within a range of ±5%, more preferably within a range of ±2%, still more preferably within a range of ±1% of its value.

As used herein, the term “pharmaceutically acceptable” is art recognized and refers to compositions, polymers and other materials and/or salts thereof and/or dosage forms which are, within the scope of sound medical judgment, suitable for use in contact with the tissues of human beings and animals without excessive toxicity, irritation, allergic response, or other problem or complication, commensurate with a reason able benefit/risk ratio.

As used herein, the terms, “pharmaceutically acceptable agent” and “pharmaceutically acceptable carrier” are used interchangeably, and are meant to have the same meaning, and are art-recognized, and refer to, for example, pharmaceutically acceptable materials, compositions or vehicles, such as a liquid (aqueous or non-aqueous) or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the composition and not injurious to a patient. In certain embodiments, a pharmaceutically acceptable carrier is non pyrogenic. Some examples of materials which may serve as pharmaceutically acceptable carriers in the context and goal of the embodiments of the invention may include: sugars such as lactose, glucose and sucrose; starches, such as corn starch and potato starch; cellulose, and its derivatives, such as sodium carboxymethyl cellulose, ethyl cellulose and cellulose acetate; powdered tragacanthin; malt, gelatin; talc.; excipients, such as cocoa butter and suppository waxes; oils such as castor oil, olive oil, peanut oil, macadamia nut oil, walnut oil, almond oil, pumpkinseed oil, cottonseed oil, sesame oil, corn oil, soybean oil, avocado oil, palm oil, coconut oil, sunflower oil, safflower oil, flaxseed oil, grapeseed oil, canola oil, low viscosity silicone oil, light mineral oil, or any combination thereof glycols, such as propylene glycol; polyols, such as glycerin, sorbitol, mannitol and polyethylene glycol; esters, such as ethyl oleate and ethyl laurate; agar, buffering agents, such as magnesium hydroxide and aluminum hydroxide; alginic acid; pyrogen-free water; isotonic saline; Ringer's solution; ethyl alcohol; phosphate buffer solutions; gums such as HP-guar; polymers; and other non-toxic compatible substances employed in pharmaceutical compositions.

It should be understood that operations, and in particular, the method steps may be shown and described as being in a sequence or temporal order, but they are not necessarily limited to being carried out, exactly as claimed, and can therefore be carried out in any particular sequence or order. In other words, as an example, if a method is claimed having at least the steps a), b) and c), a skilled artisan would understand, according to above definition, that the method may equally be performed, for example, by carrying out the method, executing the steps in the order as a), c) and b) etc.

The terms “comprises”, “comprising”, “includes”, “including”, “having” and their conjugates mean “including but not limited to”. Terms and phrases used in this application, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. Adjectives such as e.g., “conventional”, “traditional”, “known” and terms of similar meaning should not be construed as limiting the item described to a given time period, or to an item available as of a given time. But instead, these terms should be read to encompass conventional, traditional, normal, or standard technologies that may be available, known now, or at any time in the future.

Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. The presence of broadening words and phrases such as “one or more”, “at least”, “but not limited to”, or other like phrases in some instances shall not be read to mean that the narrower case is intended or required in instances, wherein such broadening phrases may be absent.

Throughout this disclosure the concentration of the lignan extract is expressed as molarity (μM). In every case the molarity of the coniferous lignan extract is calculated based on 7-hydroxymatairesinol's molarity. Concentration of betulonic- or betulinic acid is calculated as molarity of the acid in question.

It will be readily understood by one of ordinary skill in the relevant art that the present invention has broad utility and application. Although the present invention has been described and illustrated herein with referred to certain embodiments, it will be apparent to those of ordinary skill in the art that other embodiments may perform similar functions and/or achieve like results, and that the described embodiments are for illustrative purposes only.

Thus, it should be understood that various features and aspects of the disclosed of the disclosed embodiments can be combined with or substituted for one another in order to form varying modes of the disclosed invention. Many different embodiments such as variations, adaptations, modifications, and equivalent arrangements are will be implicitly and explicitly disclosed by the embodiments described herein, and thus fall within the scope and spirit of the present invention.

### EXAMPLES

### Example 1—Preparation and Extraction of a Lignan Extract with an Extraction Solution Containing Propylene Glycol

A concentrated alcohol-wood lignan extract was prepared by extracting chipped knot wood of Picea abies L. with an extraction solution composed of propylene glycol. The extract contained 7.5 wt % of the following lignans:

TABLE 1
Lignan content of an extract of Picea abies L.:

| Lignan | Amount, wt % |
|---|---|
| Hydroxymatairesimol | 5-6 |
| Secoisolariciresinol | 0.2-0.4 |
| Conidendrin | 0.2-0.4 |
| Liovil | 0.2-0.4 |
| Lariciresinol | 0.1-0.2 |
| Other lignans, including oligolignans | 0.2-1 |

In certain cases, the extract was ‘fortified’ by adding betulonic and/or betulinic acid to the extract such that the betulonic and/or betulinic acid concentration in the final composition was 10-100 μM.

### Example 2—Preparation and Extraction of a Lignan Extract with an Extraction Solution Containing Ethanol and Water

A concentrated alcohol-wood extract containing a lignan mixture presented in Table 1 was prepared by extracting chipped knot wood of Picea abies L., with an extraction solution composed of ethanol and water. The alcohol-water wood extract contained 75 wt % of the lignan mixture presented in Table 1.

In certain cases, the extract was ‘fortified’ by adding betulonic and/or betulinic acid to the extract such that the betulonic and/or betulinic acid concentration in the final composition was 10-100 μM.

### Example 3—Treatment of Androgen Independent Prostate Cancer Cells with Lignan Extracts, In Vitro, Suppresses the Proliferation of the Prostate Cancer Cells

Lignan Extract

An alcohol-water wood extract was prepared from chipped knot wood of Picea abies L by extracting the wood with ethanol-water extraction solution. The extract contained 7.5% of the lignan mixture presented in table 1 above. A composition was prepared by mixing 10 wt % of the ethanol-water wood extract with 90 wt % of 1,3-propanediol. This composition was used in the experiments below and is below called ‘the lignan extract’. A dilution series of the extract was prepared have concentrations of 24, 36, and 60, μM of the extract.

Live-Cell Imaging

The cells were cultured in either RPMI-1640 (PC-3) or DMEM media (SK-Mel-5, WM-266-4 & A2058) supplemented with 10% fetal bovine serum, 2 mM L-glutamine, 100 U/mL penicillin, and 100 μg/mL streptomycin. One day prior to the treatment, the cells were counted and plated on glass coverslips on 24-well plates (Falcon) and incubated overnight at +37° C. (5% CO₂), in order for the cells to attach to the coverslips. The following day, the cells were washed with Dulbecco's phosphate buffered saline (Bio West) and treated with 24, 36 or 60 μM of a lignan extract (Oy Granula Ab Ltd.) or solvent control (1,3-propanediol). Additionally, a control using only growth media was used. All samples were made in triplicates. The cells were imaged for 48 h with 2 h intervals (9 images per well) using IncuCyte S3 Live-Cell Analysis System (Essen BioScience). From live-cell imaging, both videos and graphs showing cell growth could be attained. The experiments were repeated at least three individual times.

Phase Contrast Imaging

Both the prostate cancer and the melanoma cell lines were cultured, plated, and treated as previously described with varying concentrations of the mixture of lignan extracts (24 μM, 36 μM, 60 μM). Phase contrast images of treated cells were taken after 24 h and 48 h incubation at +37° C. (5% CO₂) using a Zeiss Axio Vert. A1 microscope with 20× and 5× magnifications. All samples were in triplicates on 24-well plates and the experiment was repeated at least three independent times.

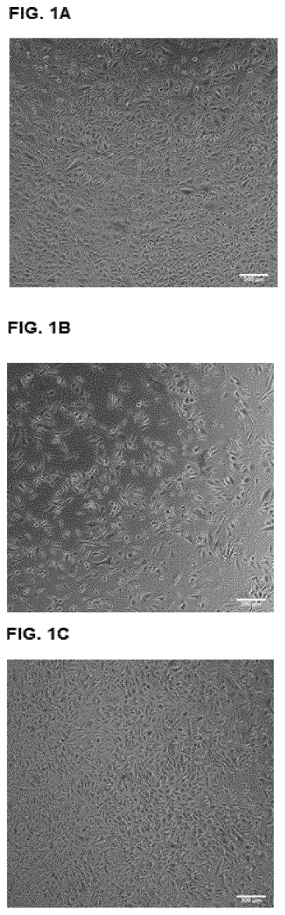

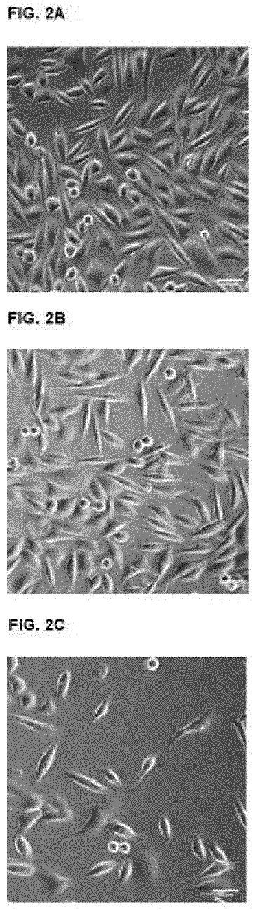

Prostate cancer cells were analyzed for cell proliferation after being treated with the lignan extract in different concentrations. When taking still images after 24 h and 48 h after treatment with 24 μM lignan extract, clear differences in the total amount of living cells in the wells could be noted for PC-3 cells (FIG. 1B). No apparent change in the cell amount between the two controls could be observed (FIG. 1A and FIG. 10). Additionally, some slight morphological changes could be seen in the cultured cells between treated and control cells (FIGS. 2A-2C). These changes were mostly a decreased abundance of actively dividing cells and an increase in senescent-like cells (FIG. 2C). Only pictures of cells treated with 24 μM lignan extract (48 h) are shown in this disclosure.

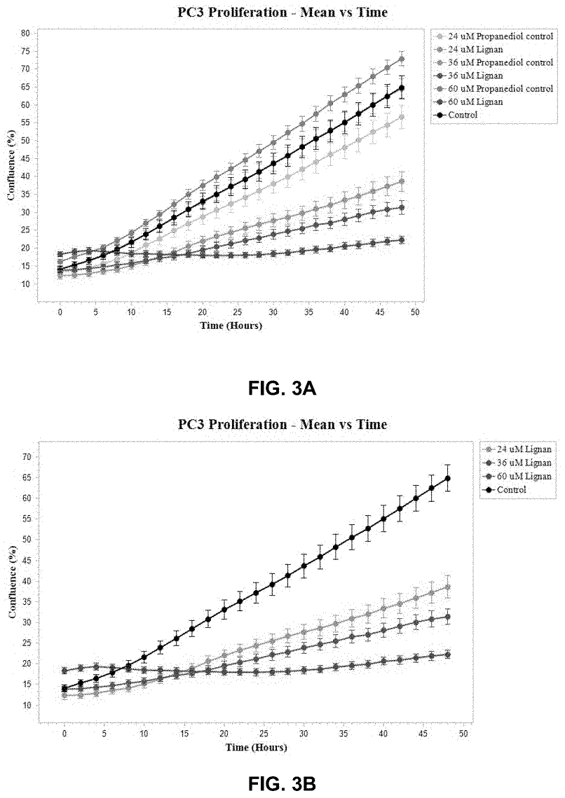

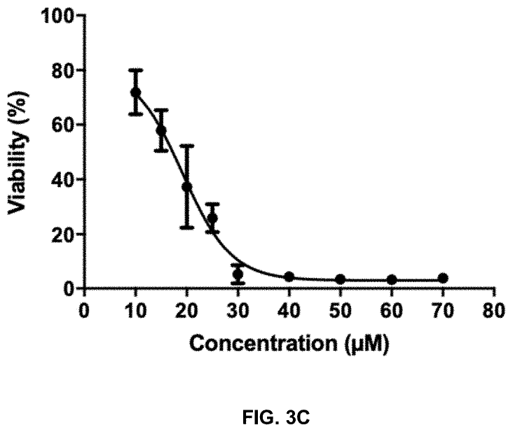

These results were confirmed when assessing the proliferation pattern of PC-3 cells, as the lignan extract had a dose-dependent response, thereby suppressing and inhibiting the growth and proliferation of the PC3, when used in a concentration ranging from 24-60 μM, as clearly depicted in FIGS. 3A and 3B.

Statistical analysis of data at incubation time 48 hours was calculated and performed with one-way ANOVA and Dunnett's multiple comparisons test using GraphPad Prism 6 software. Significant effects were found with 60 μM lignan extract treatment, where P<0.05 was considered to indicate significant differences in the data.

During the 48 h live-cell imaging, a notable suppression in the proliferation rate was found with PC-3 cells, as described hereinabove, using 24-60 μM lignan extract (FIG. 3A, blue, orange, and red graphs). Since the effect of lignans is concentration-dependent (FIG. 3A), with high concentration of 60 μM (red curve), PC-3 cells lost almost completely their capability of growth within the first 10-24 h of the treatment (FIG. 3A). With concentrations of 24 μM (orange) and 36 μM (blue), there was a clear effect on the growth rates, but the growth was not fully lost. As expected, the solvent control 1,3-propanediol did not have any effect on the PC-3 prostate cancer cells nor to their growth (light and dark grey graphs FIGS. 3A and 3B).

### Example 4—Cytotoxicity Assay of P. abies Knotwood Extract on Serum-Starved PC-3 Cells

Lignan Extract

An alcohol-water wood extract was prepared from chipped knot wood of Picea abies L by extracting the wood with alcohol-water (e.g. ethanol-water) extraction solution. The extract contained 7.5% of the lignan mixture presented in table 1 above. A composition was prepared by mixing 10 wt % of the alcohol-water wood extract with 90 wt % of 1,3-propanediol. This composition was used in the experiments below and is below called ‘the lignan extract’. A dilution series of the extract was prepared have concentrations of 10, 15, 20, 25, 30, 40, 50, 60 and 70 μM of the extract.

Cell Culture

PC-3 cells were seeded with 10 000 cells/well on a 96-well plate (Falcon) in RPMI-1640 medium (Sigma-Aldrich) supplemented with 10% fetal bovine serum, 2 mM L-glutamine, 100 U/ml penicillin, and 100 μg/ml streptomycin. The cells were let attach for 6 h prior to changing the medium to serum-free RPMI-1640 and incubating them overnight in +37° C., 5% CO₂. After overnight serum starvation, cells were treated with P. abies knotwood extract in concentrations of 0-70 μM in serum-free medium in replicate samples and incubated for 24 h. The old medium was thereafter discarded and substituted with 100 μL 10% Cell Counting Kit-8 reagent (Dojindo Molecular Technologies) diluted in serum-free RPMI-1640 medium. The samples were incubated for an additional 1 h prior to analyzing cell viability by measuring the absorbance at 450 nm using a microplate reader (Hidex Sense) according to manufacturer's protocol. Non-treated cells were used as a negative control. The experiment was repeated at least three independent times for accurate cytotoxicity analysis. The IC₅₀-value was calculated using GraphPad Prism 6 software.

FIG. 3C shows the extract inducing significant cell death in PC-3 cells under growth factor scarce condition. The graph represents mean values of viability (±SEM) from the independent experiment repeats normalized to the non-treated control sample where viability was set to 100%.

A cytotoxicity assay measuring cell viability of serum-starved PC-3 cells treated for 24 h with extract in serum-free medium revealed the IC₅₀-value to be 20.02 μM in these treatment conditions. Cell death could be observed already at low concentrations of <10 μM and the toxicity became more profound with increasing doses, indicating that the cells become sensitized to the extract when growth factors are limited. Growth factor depletion in vitro mimics to an extent the conditions in vivo, such as in a solid tumor where the amount of available circulating growth factors is poor.

### Example 5—Melanoma Cancer Cells are Less Responsive In Vitro to Lignan Extract Treatment than Androgen Independent Prostate Cancer Cells

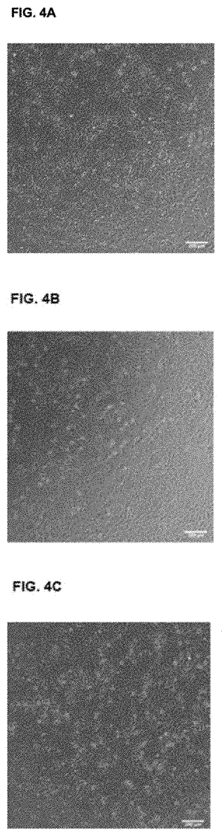

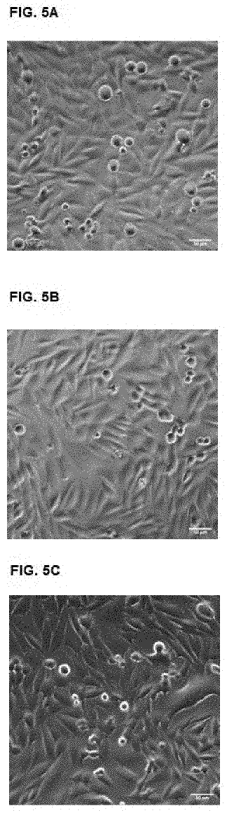

Concerning the experiments with the melanoma cancer cells, even if the lignan extract did have an effect on the proliferation rate of the cells, the effect was not as fast and strong as with prostate cancer cells. By looking at still images taken of the melanoma cell line SK-Mel-5, no clear repression of the cell growth, nor any morphological changes were observed with 24 μM lignan extract after 48 h (FIGS. 4A-4C and FIGS. 5A-5C). Similar results were obtained with A2058 and WM-266-4 cell lines (results not shown).

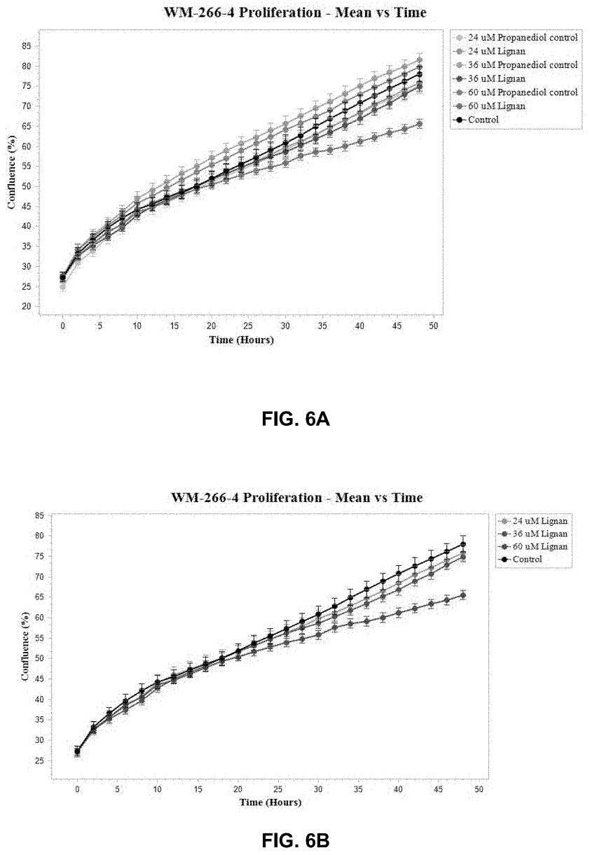

Referring to FIGS. 6A and 6B, the melanoma cell line WM-266-4 was much more resistant than PC-3 cells to the effects of the extract as seen during 48 h treatment with live-cell imaging. Nonetheless, statistically significant repression on proliferation was seen with the highest concentration (60 μM). However, the observed effects started to show first after 24 h of treatment, whereas clear suppression of cell division could be seen much earlier with PC-3 cells. Naturally, prostate cancer and melanoma cells behave in a significantly different fashion as e.g. their surface membrane receptors and signal transduction pathways vary to an extent. For these and other reasons, the two types of cancer cells are not exactly comparable to each other. Cell cycle initiation and progression is however regulated generally in a similar way in all human cell types. As with PC-3 cells, key proteins involved in cell cycle regulation seem to be affected also in WM-266-4 cells.

Again, statistical analysis of data at incubation time 48 hours was calculated and performed with one-way ANOVA and Dunnett's multiple comparisons test by using GraphPad Prism 6 software. Significant effects were found with 60 μM lignan extract treatment, where P<0.05 was considered to indicate significant differences in the data.

In conclusion, the experiments show that exposure to a lignan mixture of this disclosure results in repression of cell proliferation of various cancer cell lines generally in time and concentration dependent manner.

### Example 6—the Lignan Extract Treatment of Androgen Independent Prostate Cancer Cells Prevents their Proliferation by Arresting the Cell Cycle in G0/G1 Phase

Flow Cytometry Analysis of Cell Cycle Arrest Using Propidium Iodide Staining

PC-3 cells were seeded on 6-well plates (Falcon) with 200 000 cells/well in RPMI-1640 medium (Sigma Aldrich) supplemented with 10% fetal bovine serum, 2 mM L-glutamine, 100 U/ml penicillin, and 100 mg/ml streptomycin and incubated in +37° C. 5% CO₂ overnight. The next day cells were treated with the Picea abies lignan extract in concentrations of 0, 24 and 60 μM and incubated for an additional for 24 h. Cells were harvested thereafter by trypsinization and centrifuged down (1200 rpm for 5 min in +4° C.), after which the pellets were resuspended in Dulbecco's phosphate buffered saline (BioWest). The samples were fixed for 15 min at RT using Cytofix/Cytoperm™ (BD Bioscience). After fixation, the cells were pelleted and washed with in 1×BD Perm/Wash™ buffer (diluted in distilled water). The pellets were resuspended gently in FxCycle PI/RNase Staining Solution (ThermoFisher Scientific) and incubated for 30 min at RT in the dark in order to stain the cells. Once stained, the samples were spun down and the pellets were resuspended in FACS buffer (2% fetal bovine serum+0.4% 0.5 M EDTA in PBS) prior to analyzing them using flow cytometry (BD LSR Fortessa analyzer). At least 20 000 events per sample were collected for the analysis.

Statistical Analysis

The statistical analysis of the data was calculated by one-way ANOVA complemented with Tukey's multiple comparison test by using the GraphPad Prism 6 software. P<0.05 was considered to indicate significant differences in the data. The graphs represent the percentage of cells in each cell cycle phase from three independent experiments±standard error of the mean (SEM).

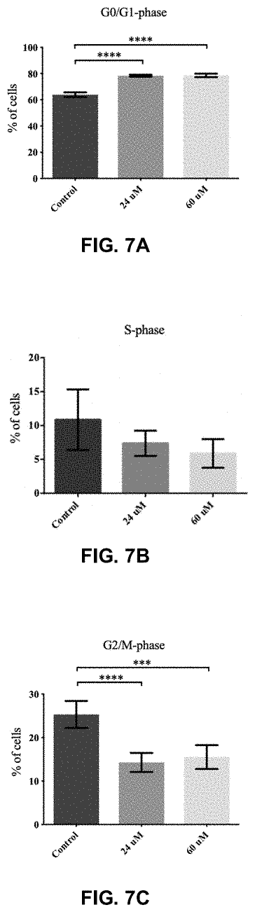

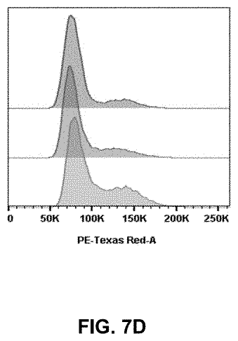

FIGS. 7 A-D show results of these experiments. Cell cycle analysis using propidium iodide staining of lignan extract treated cells showed significant differences in the proportion of cell in the various stages of the cell cycle. PC-3 cells arrested in the G0/G1-phase upon 24h treatment already with 24 μM concentrations of the extract. By arresting in G0/G1, a smaller percentage of cells were evidently operating in both the S- and the G2/M-phase, where significant differences could be noted between treated and untreated samples in the G2/M-phase. Interestingly, no clear difference was seen between 24 μM and 60 μM treatments unlike in previously performed live-cell imaging, where treatment with 60 μM caused a significant suppression in proliferation.

These results give support to an embodiment of using the extract of this invention in early phases of tumor development.

### Example 7—Low Concentrations of Betulinic Acid (BA) or Betulonic Acid (BOA) in the Extract Improve Suppression of Proliferation of Metastatic Prostate Cancer (PC-3) and Metastatic Melanoma (WM-266-4) Cells During 48 h Treatment

Lignan Extract

An alcohol-water wood extract was prepared from chipped knot wood of Picea abies L by extracting the wood with ethanol-water extraction solution. The extract contained 7.5% of the lignan mixture presented in table 1 above. A composition was prepared by mixing 10 wt % of the ethanol-water wood extract with 90 wt % of 1,3-propanediol. This composition was used in the experiments below and is below called ‘the lignan extract’. A dilution series of the extract was prepared have concentrations of 20, 40, and 60 μM of the extract. 1% DMSO was used to control for the toxicity effects of the solvent only on cell viability. Betulinic acid and betulonic acid stock solutions were diluted in DMSO. The concentration of DMSO per sample in each experiment was 1%.

Live-Cell Imaging

The cells were cultured in either RPMI-1640 (PC-3) or DMEM media (WM-266-4) supplemented with 10% fetal bovine serum, 2 mM L-glutamine, 100 U/mL penicillin, and 100 μg/mL streptomycin. One day prior to the treatment, the cells were counted and plated on 24-well plates (Falcon) and incubated overnight at +37° C. (5% CO₂). The following day, the cells were treated betulinic acid (BA) or betulonic acid (BOA) in concentrations of 0, 10, 50, 70, 100, and 150 μM. 1% DMSO was used to control for the effects of the solvent. All samples were made in triplicates. The cells were imaged for 48 h with 2 h intervals (9 images per well) using IncuCyte S3 Live-Cell Analysis System (Essen BioScience).

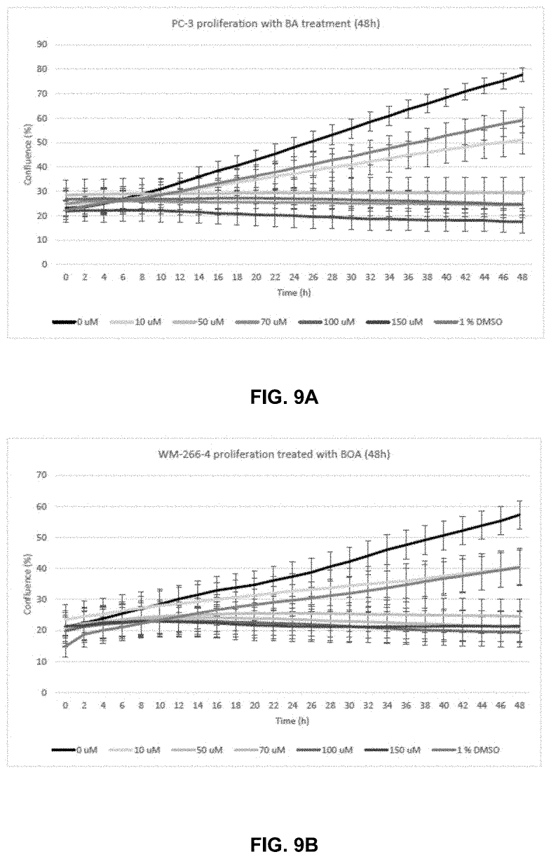

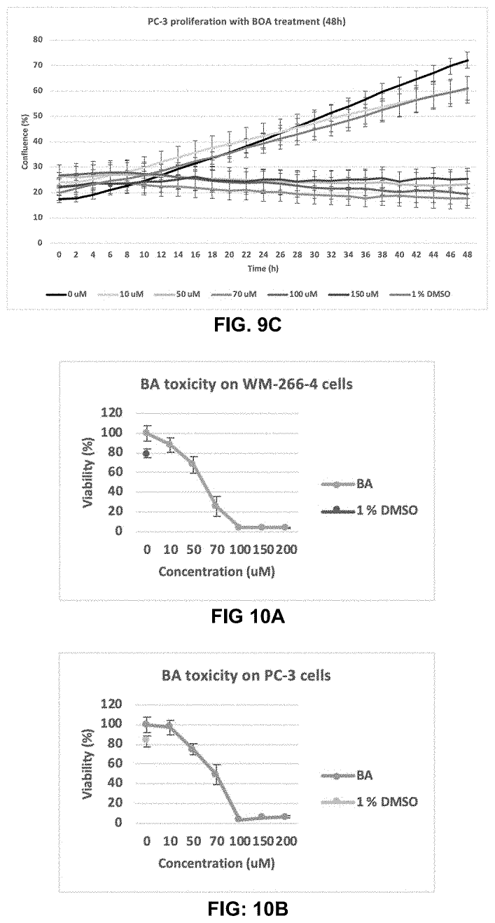

Cancer cells were analyzed for cell proliferation after being treated with BA or BOA in different concentrations for 48 h. FIG. 9A shows clearly how addition of as low as 10 uM BA clearly decreased the proliferation of PC-3 prostate cancer cells. The effect was enhanced with higher concentraionts of 50, 70, 100 and 150 μM BA. The maximum decrease of the proliferation was achieved by 50 μM BA and increase of the BA concentration beyond this did not have any further effect during the 48-hour treatment period. FIG. 9B show similar result with melanoma cells WM-266-4: addition of 50 μM BOA gave the maximal decrease in proliferation and increase of BOA concentration above this did not have any further effect. FIG. 9C shows similar results with PC-3 cells treated with BOA, where addition of 50 μM BOA gave the maximal decrease in proliferation and concentrations beyond this did not further enhance the effects.

These results were confirmed by determining viability of the cells treated with extract with various BA or BOA concentrations.

Cell Culture and Cytotoxicity Assay of BA and BOA

Cells were seeded with 5000 cells/well on a 96-well plate (Falcon) in RPMI-1640 medium (PC-3 cells) or DMEM medium (WM-266-4 cells) supplemented with 10% fetal bovine serum, 2 mM L-glutamine, 100 U/ml penicillin, and 100 μg/ml streptomycin. The cells were incubating overnight in +37° C., 5% CO₂ for the cells to adhere to the plate. The following day, cells were treated with BA or BOA (prepared from stock solutions using DMSO as solvent) in concentrations of 0-200 μM in complete medium in replicate samples and incubated for 48 h. The old medium was thereafter discarded and substituted with 100 μL 10% Cell Counting Kit-8 reagent (Dojindo Molecular Technologies) diluted in serum-free RPMI-1640 medium. The samples were incubated for an additional 1 h prior to analyzing cell viability by measuring the absorbance at 450 nm using a microplate reader (Hidex Sense) according to manufacturer's protocol. Non-treated cells were used as a negative control. 1% DMSO was used to control for effect of solvent. All samples contained 1% or less DMSO. The experiment was repeated at least three independent times for accurate cytotoxicity analysis. The IC₅₀-value was calculated using GraphPad Prism 6 software.

FIGS. 10A and B show the viability test of WM266-4 cells and PC-3 cells (respectively) treated 48 hours with various concentrations of BA. In both cases the viability of the cells dropped to close to 0% when treated with 100 uM BA for 48 hours. IC₅₀-value for BA on WM-266-4 cells was 64.67 μM, whereas for PC-3 cells the value was 67.95 μM as calculated using GraphPad Prism 6 software.

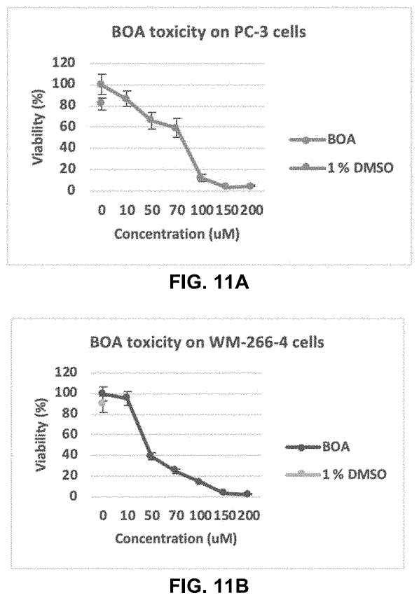

FIGS. 11A and B show the viability test of PC-3 cells and WM-266-4 cells (respectively) when treated 48 hours with various concentrations of BOA. In both case the viability decreased below 20% with 100 μM BOA treatment, but WM-266-4 cells were more responsive to lower BOA concentrations than PC-3 cells. IC₅₀-value for BOA on WM-266-4 cells was 39.83 μM, whereas for PC-3 cells the value was 78.47 μM as calculated using GraphPad Prism 6 software.

### Example 8—Synergistic Effect of Lignan Extract and Betulonic Acid or Betulinic Acid

Live-Cell Imaging

The cells were cultured in either RPMI-1640 (PC-3) or DMEM media (WM-266-4) supplemented with 10% fetal bovine serum, 2 mM L-glutamine, 100 U/mL penicillin, and 100 μg/mL streptomycin. One day prior to the treatment, the cells were counted and plated on 96-well plates (Falcon) and incubated overnight at +37° C. (5% CO₂). The following day, the cells were treated with P. abies lignan extract in concentrations of 0, 20, 40 or 60 μM in combination with betulinic acid (BA) or betulonic acid (BOA) in concentrations of 0, 10, or 30 μM. The cells were imaged for 48 h with 2 h intervals (4 images per well) using IncuCyte S3 Live-Cell Analysis System (Essen BioScience). DMSO was tested to control for the effects of the solvent used for BA and BOA stock solutions. 1,3-Propanediol was used to control for extract solvent. All samples were made in triplicates.

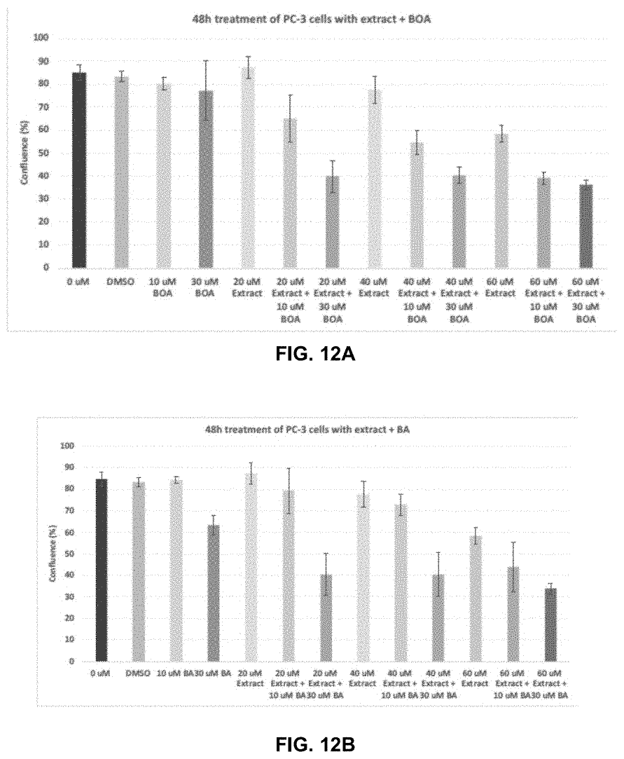

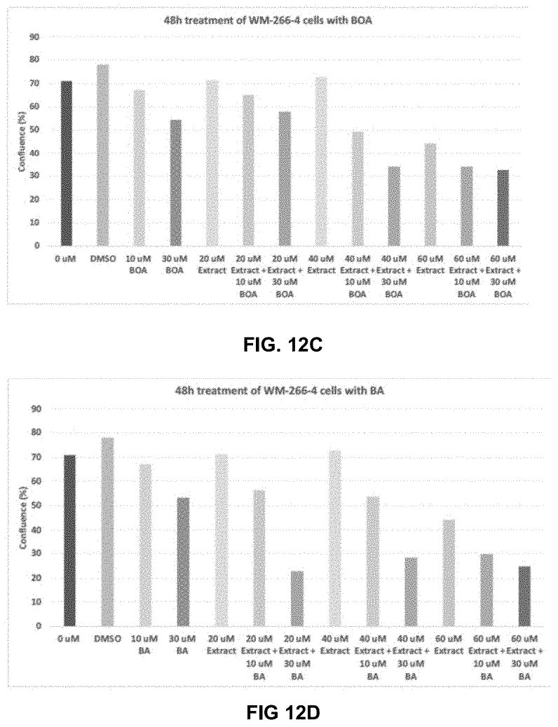

FIG. 12A illustrates end-point data from live-cell imaging experiments analyzing proliferation where prostate cancer cells were treated for 48 hours with either various concentrations of extract of Table 1, with various concentrations of BOA alone or with extract where various concentrations of BOA were added into it.

It can be seen that treatment with 40 or 60 μM extract alone decreased the proliferation in PC-3 cells. A slight enhancement of this suppression was achieved when BOA was added to the extract in concentration of 10 or 30 μM. Most dramatic decrease is achieved when the cells were treated with extract having BOA concentration of 30 μM.

FIG. 12B shows similar experiment with PC-3 prostate cancer cells treated with the extract and BA. Again, most dramatic suppression of proliferation is seen when cells are treated with extract in combination with 30 μM BA.

Thus, it can be concluded that combination of the extract with BOA or BA increases the toxic effects on PC-3 prostate cancer cells in vitro.

FIGS. 12C and 12D show similar experiments with WM-266-4 melanoma cells treated with a combination of extract and BA or BOA. The most dramatic effect with is achieved by treating the cells with 40 μM extract having BOA concentration of 30 μM (FIG. 12C). In case of BA (FIG. 12D), the most dramatic effect is achieved with 20 μM extract having added BA with concentration of 30 μM.

The lignan extract alone has weaker effect on melanoma cancer cell growth than it has on prostate cancer cell growth as discussed in Example 5 above. However, adding BA or BOA into the extract synergistically suppresses melanoma cell proliferation in a concentration-dependent manner.

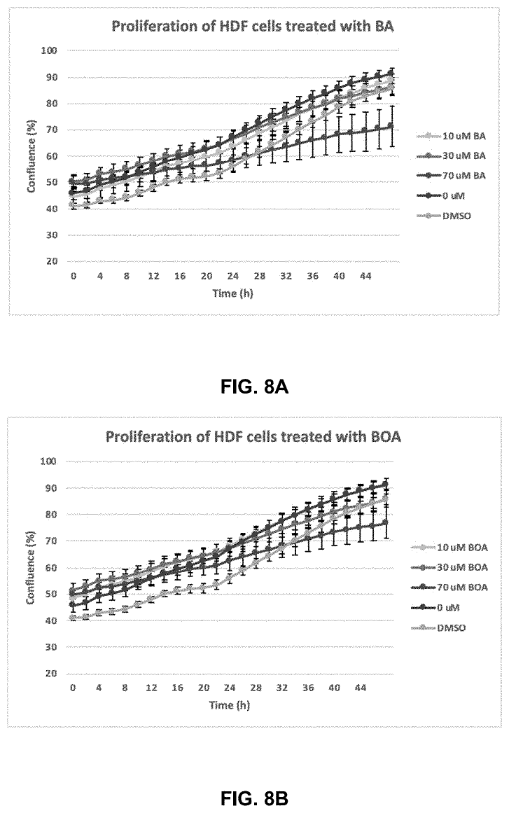

The experiments show that combining extract with low concentrations of BA or BOA sensitizes the cells for cell death or at least inhibit proliferation as is seen in the end-point data after 48 hours treatment for both PC-3 and WM-266-4 cells. Importantly, as is shown in FIGS. 8A and B neither BA or BOA had any significant effects on HDF cells in the concentrations used, indicating that the compounds are non-toxic to normal cells while being selectively toxic to melanoma and prostate cancer cells.

### Example 9—Testimonial Examples of Effects of Lignan Extract Treatment on Tumor Growth

A composition of lignan extract of Picea abies L. having lignan content of table 1 was administered orally daily to individuals having been diagnosed with tumor growth. 7-Hydroxymatairesinol concentration in the composition was 6.7 mg/0.1 ml. The daily dosage was 15-30 droplets per day and each droplet was about 10 microliters. The extract was taken orally three times per day 5-10 droplets at a time.

Table 2 below illustrates the effects of the lignan extract treatment:

| | Diagnosed | Lignan | | |
|---|---|---|---|---|
| Individual | condition | treatment | Outcome | Outcome |
| Female | Breast cancer; | 20-30 | Lump | No cancer |
| human | a 6 mm lump | droplets | completely | diagnosis |
| | in breast | per day | disappeared | after next |
| | | | | follow up |
| Female | Colon cancer; | 30 droplets | Regained | No cancer |
| human | Kidney | per day | appetite | diagnosis |
| | malfunction | | after 2 | after |
| | | | weeks of | 15 months of |
| | | | treatment | treatment |
| Female | Brain tumor | 30 droplets | Energized | No tumor |
| human | | per day | and lost | found after |
| | | | headaches | 180 days |
| | | | after a few | of treatment |
| | | | days of | |
| | | | treatment | |
| Male | Enlarged | 30 droplets | Reduced need | Loss of a |
| human | prostate | | to urinate | feeling of |
| | | | during | painful |
| | | | nighttime | pressure |
| | | | after a few | |
| | | | days of | |
| | | | treatment | |
| Female | Unknown | About 15 | Size of the | Lumps |
| Dog | cancer with | droplets | lump reduced | disappeared |
| | lumps in | per day | to about half | by next |
| | the chest | | within weeks | appointment |

The above results are illustrative to show that the extract is effective to prevent cancer cell proliferation also in vivo. Cancer types that seem to be affected comprise at least breast cancer, brain cancer, and colon cancer. The extract is effective generally in mammals. There is also evidence that administering the composition topically (spray or ointment) heals burning injuries on skin caused by radiation treatment. Topical administration can also be used as a preventive measure for injuries caused by radiation treatments.

What is claimed is:

1. A composition for suppressing mammalian cancer cell proliferation or growth, the composition being a solution comprising an alcoholic lignan extract or a mixture of alcoholic lignan extracts from one or more of coniferous species selected from genus Picea, Abies, and Pinus, and optionally betulonic or betulinic acid, wherein the lignan extract or mixture of lignan extracts comprises at least the following lignans: 7-hydroxymatairesinol, conidendrin, lariciresinol, liovil and secoisolariciresinol or their geometric isomers or stereoisomers and a fraction containing various oligolignans.

2. The composition of claim 1, wherein the alcoholic extract or the mixture of alcoholic lignan extract comprises lower mono- or/and dialcohols, such as ethanol or propanediol.

3. The composition of claim 1, wherein the composition comprises 5-50 wt % of the lignan extract and concentration of said lignans in the lignan extract is:
7-hydroxymatairesinol 70-80 wt %;
conidendrin 3-8 wt %;
lariciresinol 1-4 wt %;
liovil 2-5 wt %;
secoisolariciresinol 3-7 wt %; and
other lignans 0-3 wt %.

4. The composition according to claim 1, wherein the composition additionally comprises one or more lignans at a range of 0-10 wt % selected from the group consisting of pinoresinol, nortrachelogenin, matairesinol, juvabiones, their geometric isomers or stereoisomers, and oligolignans.

5. The composition according to claim 1, wherein the composition is for suppressing cancer cell proliferation and/or growth in an early phase in a mammal having diagnosed with melanoma, a brain cancer tumor, a colon cancer tumor, a breast cancer tumor, or a prostate cancer tumor.

6. The composition of claim 1, wherein the composition comprises 10-150 μM betulinic or betulonic acid.

7. The composition of claim 6, wherein the composition is effective in suppressing mammalian melanoma or prostate cancer cell proliferation.

8. The composition according to claim 1, wherein the composition is effective in suppressing cancer cell proliferation and/or growth in combination with chemotherapy and/or radiation.

9. A method of treating a mammal diagnosed with cancer by suppressing cancer cell proliferation and/or growth by daily oral administration of a composition comprising a lignan extract or a mixture of lignan extracts from one or more of coniferous species selected from genus Picea, Abies, and Pinus and optionally betulinic acid or betulonic acid preferably from bark of Betula sp., wherein said extract or mixture of extracts comprises at least the following lignans: 7-hydroxymatairesinol, conidendrin, lariciresinol, liovil and secoisolariciresinol or their geometric isomers or stereoisomers.

10. The method according to claim 9, wherein the composition comprises 5-50 wt % of the lignan extract and concentration of said lignans in the lignan extract or the mixture of extracts is 7-hydroxymatairesinol 70-80 wt %; conidendrin 3-8 wt %; lariciresinol 1-4 wt %; liovil 2-5 wt %; secoisolariciresinol 3-7 wt %; and other lignans 0-3 wt %.

11. The method according to claim 9, wherein the cancer cells are selected from the group consisting of melanoma cells, brain cancer cells, colon cancer cells, breast cancer cells, and prostate cancer tumor.

12. The method according to claim 9, wherein the composition comprises of 10-150 μM betulinic acid or betulonic acid.

13. The method according to claim 12, wherein the cancer cells are melanoma or prostate cancer cells.

14. The method according to claim 9, wherein the extract is administered in an amount that contains 20-250 mg, more preferably 50-200 mg and most preferably 100-200 mg of 7-hydroxymatairesinol.

15. The method according to claim 9, wherein the method is used in combination with radiation- or chemotherapy.

16. A method for suppressing proliferation of prostate and melanoma cancer cells in vitro, comprising a step of adding on the prostate or melanoma cell culture a composition comprising a lignan extract or a mixture of lignan extracts from one or more of coniferous species selected from genus Picea, Abies, and Pinus and optionally betulonic acid or betulinic acid, and comprising at least the following lignans: 7-hydroxymatairesinol, conidendrin, lariciresinol, liovil and secoisolariciresinol or their geometric isomers or stereoisomers.

17. The method according to claim 16, wherein the composition comprises 5-50 wt % of the lignan extract and concentration of said lignans in the lignan extract or the mixture of extracts is 7-hydroxymatairesinol 70-80 wt %; conidendrin 3-8 wt %; lariciresinol 1-4 wt %; liovil 2-5 wt %; secoisolariciresinol 3-7 wt %; and other lignans 0-3 wt %.

18. The method of claim 16, wherein the composition comprises the lignan extract or composition of the lignan extracts and betulonic acid or betulinic acid, and suppressing effect of the composition is synergistic as compared to an effect of the extract or mixture of extracts and betulinic or betulonic acids separately.
